(19) United States
(12) Plant Patent Application Publication
(10) Pub. No.: US 2019/0297761 P1
(43) Pub. Date: Sep. 26, 2019

(54) Rhododendron L. Plant Named 'Strawberry Sundae'

(50) ;

(71) Applicant: Akatsuka Garden Co., Ltd., Tsu City (JP)

(72) Inventor: Yukio Kurabayashi, Tsu City (JP)

(21) Appl. No.: 15/932,619

(22) Filed: Mar. 23, 2018

(51) Int. Cl. A01H 6/36

(52) U.S. Cl. PLT/238

(57) ABSTRACT

‘Strawberry Sundae’ is a new variety of rhododendron. This variety was produced by the hybridization of the ‘Coral Moon’ variety with the ‘Atlas’ variety in 2011. The plant may be used, e.g., as a flowering plant for outdoor cultivation, or as a potted plant. ‘Strawberry Sundae’ is visibly appealing, and produces flowers comprising a corolla that comprises vivid red-purple and light purple-pink colors.

Plant name and variety denomination: Latin name of the genus and species of the plant claimed: Rhododendron L. The claimed plant is a hybridization of different species. The parental lines of the claimed plant resulted from the hybridization of different species. Common names of the claimed plant's species: Rhododendron, Azalea, Azaleodendron. Variety denomination: ‘Strawberry Sundae’.

### BACKGROUND OF THE INVENTION

Rhododendron is a genus of woody plants in the Ericaceae family, either evergreen or deciduous, and found mainly in Asia, although it is also widespread throughout the highlands of the Appalachian Mountains of North America. Most species have brightly colored flowers that bloom from late winter through to early summer.

Both species and hybrid rhododendrons (including azaleas) are used extensively as ornamental plants in landscaping in many parts of the world, including both temperate and subtemperate regions. Many species and cultivars are grown commercially for the nursery trade.

Rhododendrons are often valued in landscaping for their structure, size, flowers, and the fact that many of them are evergreen. Azaleas are frequently used around foundations and occasionally as hedges, and many larger-leafed rhododendrons lend themselves well to more informal plantings and woodland gardens, or as specimen plants.

### BRIEF SUMMARY OF THE INVENTION

‘Strawberry Sundae’ is a new variety of rhododendron (more specifically azalea, still more specifically azaleodendron) bred by pollinating variety ‘Coral Moon’ with pollen from variety ‘Atlas’ in 2011. The plant may be used, e.g., as a flowering plant for outdoor cultivation or as a potted plant.

‘Strawberry Sundae’ is visibly appealing, and produces flowers comprising a corolla that has spots and vivid red-purple and light purple-pink colors.

‘Coral Moon’ rhododendron (unpatented) is a rhododendron variety that originated in Japan in 2003. ‘Coral Moon’ was produced by the hybridization of the ‘Rouge Magic’ (unpatented) variety with the ‘Wedding Bouquet’ (unpatented) variety. This variety has a small tree size in the shape of a dome. The flowers of this variety have a small diameter with a bright pinkish-red color. There are about 15 pieces of blossom flower in the shape of a small dome.

‘Atlas’ rhododendron (unpatented) is a rhododendron variety that originated in Japan in 2005. ‘Atlas’ was produced by the hybridization of the ‘Helios’ variety with the ‘Blue Eye’ variety. This variety has a medium tree size and spreading branches. The flowers of this variety have a medium diameter and a pale red color. There are about 15 pieces of flower blossoms in the shape of a pyramid.

‘Strawberry Sundae’ was asexually reproduced by tissue culture in Japan at Kamiogoso2080, Anocho Toshima, Tsu-shi, Mie, 514-2322. Specifically, propagation was carried out by taking a growing point from a lateral bud of a sprout and cultivating the growing point, without use of callus. Plants propagated from a ‘Strawberry Sundae’ plant by asexual or vegetative means are uniform and stable.

### BRIEF DESCRIPTION OF THE DRAWINGS

FIG. 1 is a photograph of a ‘Strawberry Sundae’ plant body.

FIG. 2 is a photograph depicting the full view of young ‘Strawberry Sundae’ plants being cultivated in a divided tray.

FIG. 3 is a photograph showing an enlarged view of a ‘Strawberry Sundae’ flower.

FIG. 4 is a photograph of a dissected ‘Strawberry Sundae’ flower.

FIG. 5 is a photograph of flowers of comparative variety Mrs. Fuji.

FIGS. 6A-D are photographs showing measurements of various different parts of ‘Strawberry Sundae’ plants. The units shown are centimeters.

FIG. 7 is a photograph of the plant body (including flowers) of parental variety ‘Atlas’.

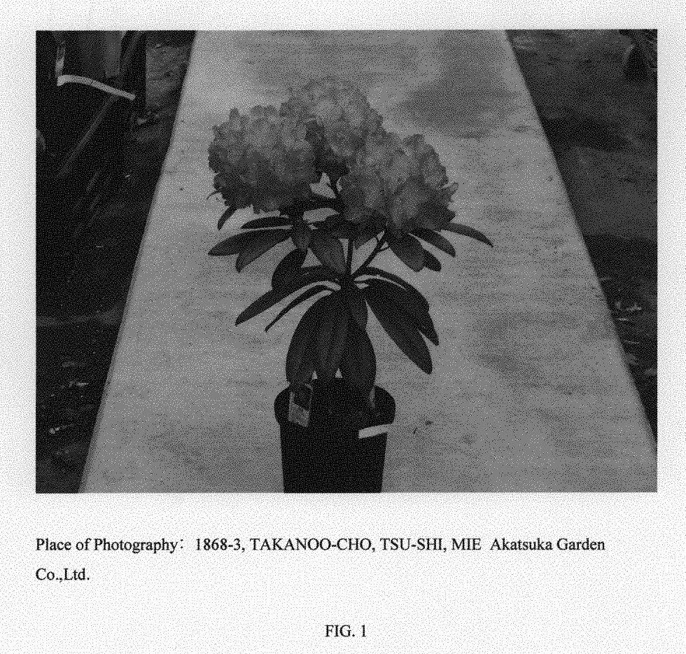

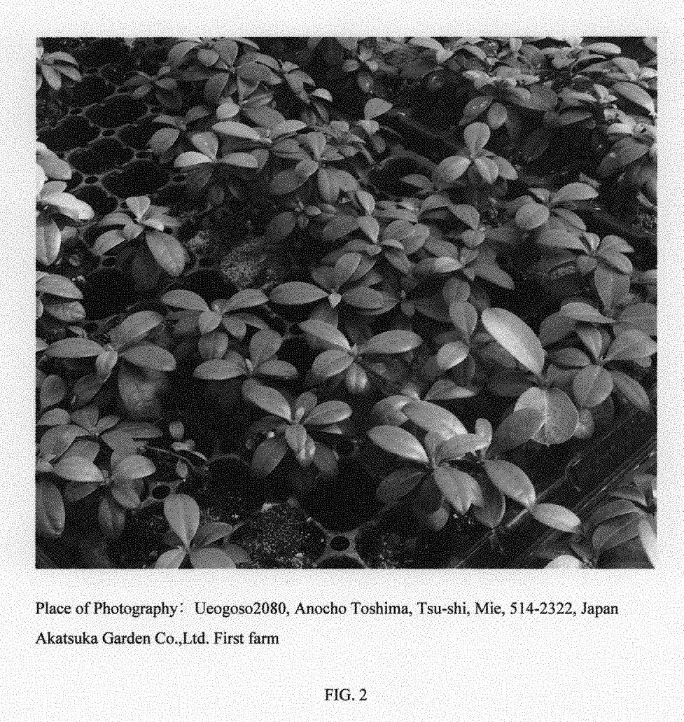

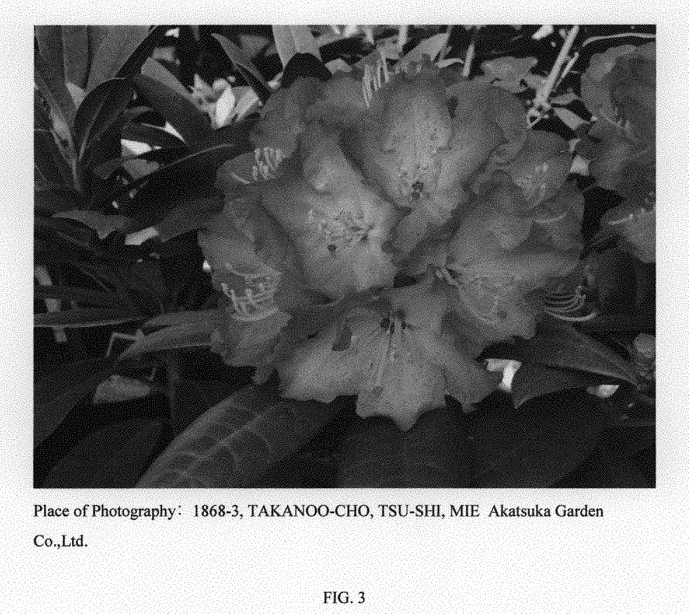

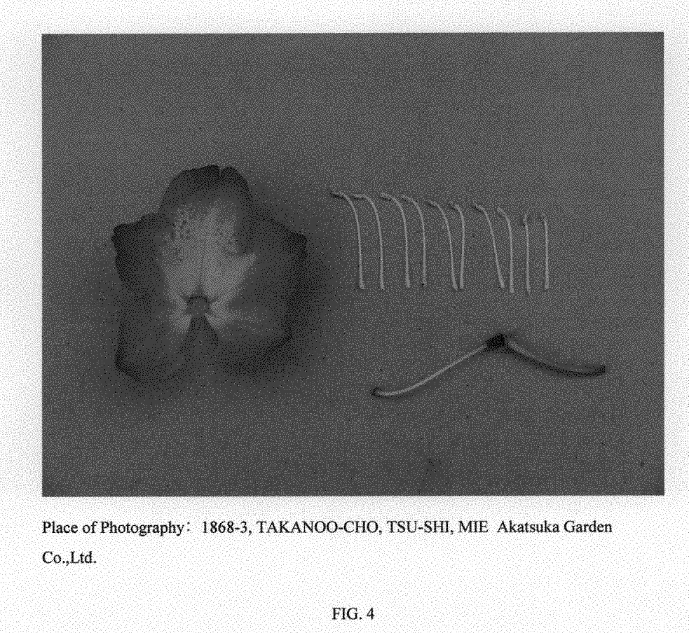

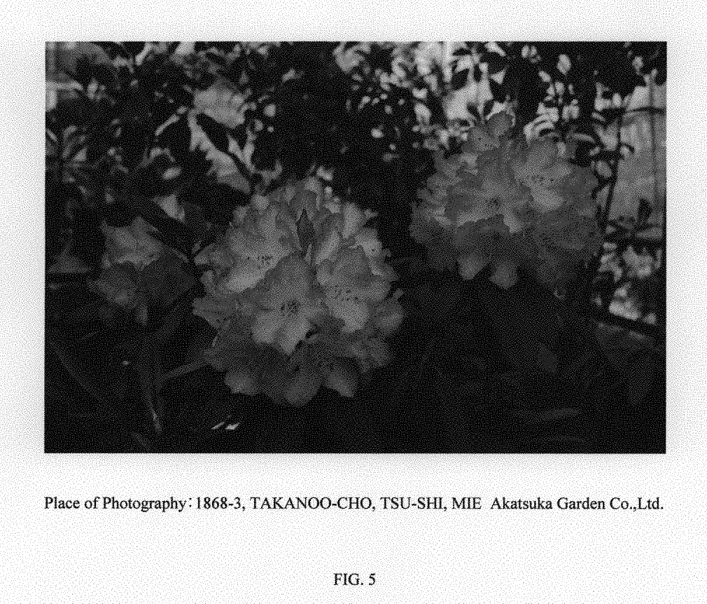

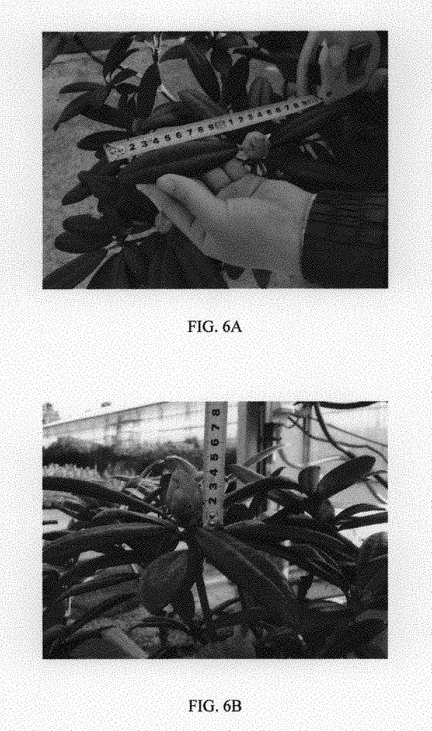

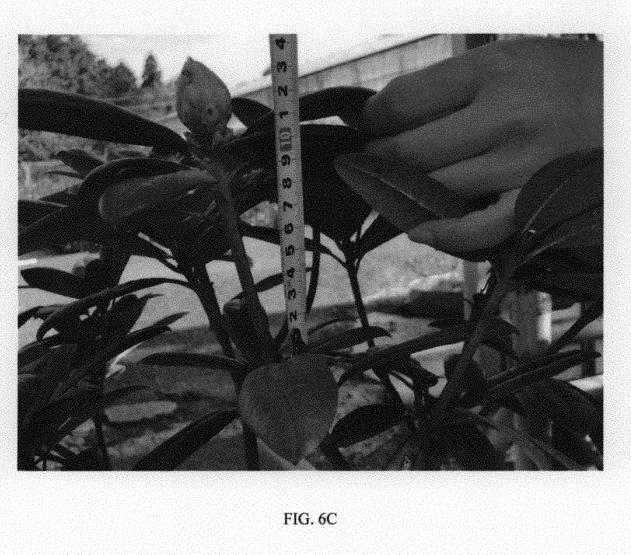

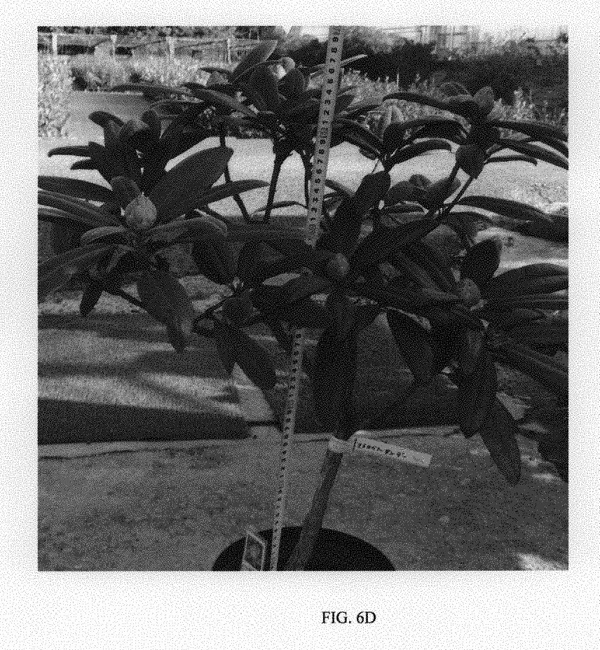

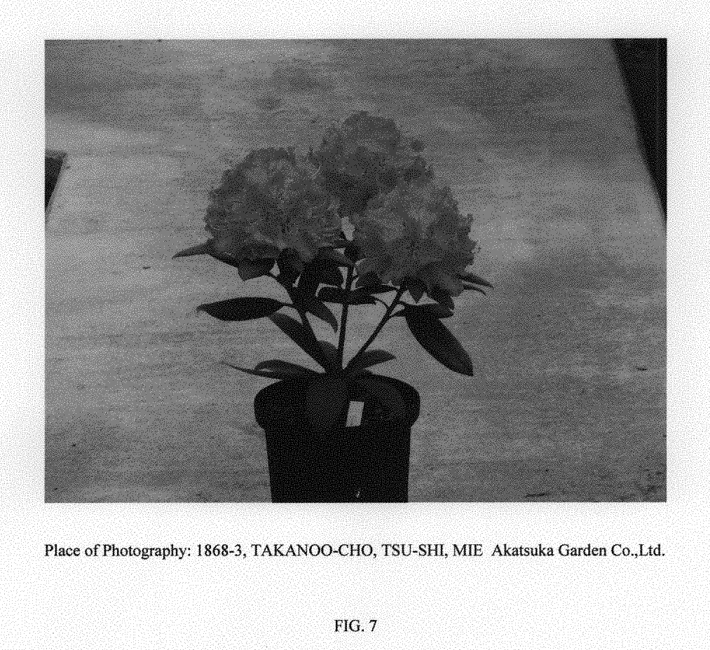

### DETAILED BOTANICAL DESCRIPTION OF THE PLANT

Variety: ‘Strawberry Sundae’

Species of the plant claimed: Rhododendron L. The claimed plant is a hybridization of different species. The parental lines of the claimed plant resulted from the hybridization of different species.

Common names of the claimed plant's species include: Rhododendron, Azalea, Azaleodendron

Characteristics of ‘Strawberry Sundae’ provided herein were observed when the plant was cultivated in the open with rain cover at Akatsuka Garden Co., Ltd., “first farm” having an address at Ueogoso2080, Anocho Toshima, Tsu-shi, Mie, 514-2322, Japan. Descriptive terms used in Tables 1, 2, and 3 herein are consistent with the Japanese Examination Standard for Rhododendron/Azalea (Rhododendron L.), TG/42/6 1995-10-20, pages 1-17, available at www.hinshu2.maff.go.jp/info/sinsakijunikijun/1543.pdf, the entire contents of which are incorporated herein by reference.

Properties and characteristics of ‘Strawberry Sundae’ are described in Table 1.

TABLE 1
Properties and characteristics of ‘Strawberry Sundae’

| | | | Class Value |
|---|---|---|---|
| | | | (Numeric |
| Charact- | | | Data) of |
| eristic | | | ‘Strawberry |
| No. | Description | State of Expression | Sundae’** |
| 1 | Plant: persistence of leaves | evergreen | 3 |
| 24 | Flower: shape | open funnel-shaped | 2 |
| 25 | Flower: diameter | medium | 5 |
| 30 | Corolla lobe: number of | two | 2 |
| | color on upper side | | |
| | (excluding markings of the | | |
| | throat) | | |
| 31 | Corolla lobe: main color on | Japan color standard | Not |
| | upper side | for horticultural plants | Applicable |
| | | (JHS) Color Chart No. | |
| | | 9507 (vivid red-purple) | |
| 32 | Corolla lobe: secondary | JHS Color Chart No. | Not |
| | color on upper side | 9501 (light purple-pink) | Applicable |
| 35 | Corolla lobe: | medium | 5 |
| | conspicuousness of | | |
| | markings of the throat | | |
| 36 | Corolla lobe: type of | spots not touching each | 1 |
| | markings | other | |
| 42 | Time of beginning of | late* | 7 |
| | flowering | | |

*Late March to early April, e.g., April 1^{st}, when grown at Akatsuka Garden Co., Ltd., “first farm” having an address at Ueogoso2080, Anocho Toshima, Tsu-shi, Mie, 514-2322, Japan.
**Numerical Values in this column correspond to the “state” in the Table of Characteristics of the Japanese Examination Standard for Rhododendron/Azalea (Rhododendron L.), TG/42/6 1995-10-20, pages 1-17, available at www.hinshu2.maff.go.jp/info/sinsakijun/kijun/1543.pdf.

When grown in Tsu-shi, Mie, 514-2322, Japan (or in an areas with a similar latitude and climate) ‘Strawberry Sundae’ should be planted between Early October to late March. In this region, flowering time is from late March to early April. Locations suitable for the growth of ‘Strawberry Sundae’ included mesic areas with good drainage and a soil pH of 5.0 to 5.5.

A variety that is similar to ‘Strawberry Sundae’ is ‘Mrs. Fuji’. Characteristics of ‘Strawberry Sundae’ and ‘Mrs. Fuji’ are compared in Table 2. Flowers of ‘Mrs. Fuji’ are shown in FIG. 5.

TABLE 2
Distinguishable Characteristics between
‘Strawberry Sundae’ and ‘Mrs. Fuji’

| Denomination | | State of | State of |
|---|---|---|---|
| of Similar | Description of | Expression in | Expression in |
| Variety | Characteristics | Similar Variety | Candidate Variety |
| Mrs. Fuji | Corolla lobe: main | 9204 (vivid | 9507 (vivid red- |
| | color on upper side | purple-pink) | purple) |

A comparison between features of ‘Strawberry Sundae’ and its parental varieties is provided in Table 3.

TABLE 3
Description of Physical Differences between Parental Plants

| | Characteristic of | | |
|---|---|---|---|
| Variety | flower | Shape of tree, etc. | Origin |
| Claimed | Large diameter with | Medium size of | First hybridized |
| Variety: | edge colored, dark | tree in the shape | in 2011. |
| ‘Strawberry | vivid red-pink and 20 | of dome. | Flowering first |
| Sundae’ | pieces of blossom | Gloss of leaves | observed in |
| | flower in the shape | is strong. | 2014. |
| | of dome. | | |
| Mother | Small diameter flow- | Small size of | Original variety |
| Variety: | ers, bright pinkish- | tree in the | obtained |
| ‘Coral | red. 15 | shape of a dome. | by hybridiz- |
| Moon’ | pieces of blossom | | ation of ‘Rouge |
| | flower in the shape of | | Magic’ variety |
| | small dome. | | with ‘Wedding |
| | | | Bouquet’ vari- |
| | | | ety in 2003. |
| Father | Medium diameter | Medium size | Original variety |
| Variety: | flower, pale red. | of tree | obtained by |
| ‘Atlas’ | Around 15 pieces of | having spreading | hybridization |
| | blossom flower in the | branch. | of ‘Helios’ vari- |
| | shape of pyramid | | ety with ‘Blue |
| | | | Eye’ variety in |
| | | | 2005. |

It will be understood that the average size of the plant may vary with location, season, nutrition, irrigation, etc.

What is claimed is:

1. A new and distinct Rhododendron L. plant named ‘Strawberry Sundae’ as illustrated and described.
